(19) United States
(12) Patent Application Publication
(10) Pub. No.: US 2015/0374639 A1
(43) Pub. Date: Dec. 31, 2015

(54) ENCAPSULATION OF HYDROPHILLIC ANTIRETROVIRAL DRUGS IN NANOCARRIERS

(71) Applicant: Creighton University, Omaha, NE (US)

(72) Inventors: Christopher J. Destache, Omaha, NE (US); Abhijit Date, Baltimore, MD (US)

(21) Appl. No.: 14/792,288

(22) Filed: Jul. 6, 2015

Related U.S. Application Data
(63) Continuation-in-part of application No. 14/422,747, filed on Feb. 20, 2015.
(60) Provisional application No. 61/697,083, filed on Sep. 5, 2012.

(51) Int. Cl. A61K 9/51

(52) U.S. Cl. CPC A61K 9/5153; A61K 9/51; A61K 9/5192

(57) ABSTRACT

An encapsulated hydrophilic antiretroviral drug including a biodegradable polymeric nanoparticle and a process for fabricating biodegradable polymeric nanoparticles to encapsulate hydrophilic antiretroviral drugs are described. In an implementation, an encapsulated hydrophilic antiretroviral drug including a biodegradable polymeric nanoparticle includes a hydrophilic antiretroviral drug; and a biodegradable polymer polymeric nanoparticle that encapsulates the hydrophilic antiretroviral drug to form a nano-sized encapsulated hydrophilic antiretroviral drug.

### CROSS-REFERENCE TO RELATED APPLICATIONS

The present application claims the benefit under 35 U.S.C. §120 of U.S. application Ser. No. 14/422,747, filed Feb. 20, 2015, and titled “POLYMERIC NANOPARTICLES IN A THERMOSENSITIVE GEL FOR COITAL-INDEPENDENT VAGINAL PROPHYLAXIS OF HIV,” which claims the benefit of International Application No. PCT/US2013/052829, filed Jul. 31, 2013, and titled “POLYMERIC NANOPARTICLES IN A THERMOSENSITIVE GEL FOR COITAL-INDEPENDENT VAGINAL PROPHYLAXIS OF HIV,” which claims the benefit under 35 U.S.C. §119(e) of U.S. Provisional Application Ser. No. 61/697,083, filed Sep. 5, 2012, and titled “POLYMERIC NANOPARTICLES IN A THERMOSENSITIVE GEL FOR COITAL-INDEPENDENT VAGINAL PROPHYLAXIS OF HIV.” U.S. Provisional Application Ser. No. 62/041,172, International Application No. PCT/US2013/052829, and U.S. application Ser. No. 14/422,747, are herein incorporated by reference in their entireties.

### FEDERALLY SPONSORED RESEARCH OR DEVELOPMENT

This invention was made with support under NIAID grant number R56AI095115-01 awarded by the National Institute of Allergy and Infectious Diseases. The Government has certain rights in this invention.

### BACKGROUND

Prevention of HIV-1 infection to reduce the number of newly infected patients is an international priority. Various modalities such as male circumcision, prophylactic HIV vaccines, vaginal microbicides, and oral pre-exposure prophylaxis have been explored to prevent sexual contraction of HIV. Prevention of HIV infection by using antiretroviral agents as vaginal microbicides has received more attention in recent years.

Standard antiretroviral therapy (ART) can include the combination of antiretroviral (ARV) drugs to maximally suppress and treat infection by retroviruses. Antiretroviral drugs inhibit the reproduction of retroviruses, which can include viruses composed of RNA rather than DNA. The best known retrovirus includes human immunodeficiency virus (HIV), the causative agent of AIDS.

### SUMMARY

An encapsulated hydrophilic antiretroviral drug including a biodegradable polymeric nanoparticle and a process for fabricating biodegradable polymeric nanoparticles to encapsulate hydrophilic antiretroviral drugs are described. In an implementation, an encapsulated hydrophilic antiretroviral drug including a biodegradable polymeric nanoparticle includes a hydrophilic antiretroviral drug; and a biodegradable polymer polymeric nanoparticle that encapsulates the hydrophilic antiretroviral drug to form a nano-sized encapsulated hydrophilic antiretroviral drug.

In an implementation, a process for fabricating biodegradable polymeric nanoparticles to encapsulate hydrophilic antiretroviral drugs includes dissolving at least one of a biodegradable polymer, a poloxamer, or a hydrophilic antiretroviral drug to form a solution; forming a homogenous phase of the solution; emulsifying the solution to form an oil-in-water emulsion; and evaporating the oil-in-water emulsion to form an encapsulated hydrophilic antiretroviral drug including a biodegradable polymeric nanoparticle.

In an implementation, a process for fabricating biodegradable polymeric nanoparticles to encapsulate tenofovir disoproxil fumarate includes dissolving a polylactic-co-glycolic acid (PLGA), a poloxamer, and tenofovir disoproxil fumarate in ethyl acetate and ethanol to form a solution; forming a homogenous phase of the solution by using an orbital shaker at approximately 40° C.; adding dissolved docusate sodium to the solution; emulsifying the solution with water using an ice bath and a sonicator to form an oil-in-water emulsion; and evaporating the ethyl acetate and the ethanol from the oil-in-water emulsion to form a biodegradable polylactic-co-glycolic acid (PLGA) encapsulated tenofovir disoproxil fumarate nanoparticle.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### DRAWINGS

The detailed description is described with reference to the accompanying figures. The use of the same reference numbers in different instances in the description and the figures may indicate similar or identical items.

FIG. 1 is an image illustrating an electron microscope photograph of nanoparticles, in accordance with an example implementation of the present disclosure.

FIG. 2 illustrates Epivaginal™ tissue viability. Vaginal tissue explants (n=3) were subjected to TDF solution in TMS gel, TDF NP in TMS gel, and blank NP in TMS gel. 1% triton and lubricant jelly were positive and negative controls. Tissue viability was determined using MTT assay methodology at 1, 4, and 24 h of incubation in triplicate for each experiment. Significant cytotoxicity was found (ANOVA) for 1% triton (p<0.01) at all time points.

FIG. 3 illustrates an HIV-1 prevention experimental design. NSG mice underwent humanization procedure and were allowed to reconstitute their human immune system for a minimum of 9 weeks and randomly divided into Ctr and Rx groups. Hu-mice in the Rx group received TMS gel containing 0.1% or 0.5% w/v TDF NPs intravaginally. HIV-1 (two strains 2.4×10⁵ TCID₅₀ were intravaginally administered at either 4 or 24 h after TDF NP administration. At weekly intervals, blood was withdrawn to monitor for PVL using qRT-PCR. At euthanasia, FRT and spleen were harvested and fixed in 4% paraformaldehyde for detection of vRNA using ISH.

FIG. 4 illustrates a TDF NP in TMS gel prevention curve. Control Hu-mice (n=8) received blank TMS gel and were 100% infected within 14 days PI. All Hu-mice receiving TDF (0.1%) NP (n=3) and challenged 4 h later or receiving TDF (0.5%) NP (n=4) and challenged 24 h later were protected from HIV-1 infection (p<0.05). Hu-mice receiving TDF (0.1%) NP (n=3) and challenged 24 h later were 33% protective from HIV-1, respectively.

FIG. 5 illustrates results from short-term in vitro prophylaxis studies. TZM-bl cells (1×10⁴ cells/well) were incubated for 24 h with different concentrations of tenofovir (TFV) solution, TDF solution, or TDF NPs. Media was removed, replaced and HIV-1_{NL4-3} (25 mL) was added to the cell for 4 h. Cells were lysed and luminescence compared to control TZM-bl cells without treatment. Significant protection of TZM-bl cells was found (*p<0.05; **p<0.01) when cells were treated 631 with TDF solution and TDF NPs.

FIG. 6 is a flow diagram illustrating a process in an example implementation for fabricating the encapsulation of hydrophilic antiretroviral drugs using nanocarriers, in accordance with an example implementation of the present disclosure.

### DETAILED DESCRIPTION

### Overview

Worldwide, nearly half of all individuals living with HIV are now women, who acquire the HIV virus largely by heterosexual exposure. Many women, because of limited economic options and gender inequality, cannot reliably negotiate sexual encounters, leaving them vulnerable to unwanted pregnancy and sexually transmitted infections (STIs), including HIV. In the absence of an effective vaccine, topical microbicide formulations, which are applied vaginally or rectally, represent an attractive solution to stop HIV transmission. However, clinical trials focusing on vaginal prophylaxis of HIV using topical microbicides have shown mixed results. Several topical microbicides such as BufferGel™, PRO 2000, and Carraguard™ have failed to show efficacy in clinical trials whereas coitally-dependent administration of 1% tenofovir gel has shown some success. Conversely, the VOICE trial employing a coitus-independent, once daily administration of 1% tenofovir gel was halted due to lack of efficacy. The VOICE trial setback has prompted investigators to examine alternatives. Therefore, there is a need for new anti-viral drugs that provide sustained delivery of anti-viral therapy for the prevention of sexual HIV transmission but can be used coitally-independent.

Hydrophilic antiretroviral drugs, such as tenofovir disoproxil fumarate, abacavir sulfate, and/or atazanavir sulfate are difficult to encapsulate into a nanocarrier. Typically, synthesis of lipophilic derivatives of these drugs is required to improve their encapsulation into nanocarriers. However, the synthesized lipophilic derivatives of these drugs would have to undergo rigorous regulatory process of drug approval. A process that can enable encapsulation of these hydrophilic drugs into nanocarriers without any chemical modification would be of great importance.

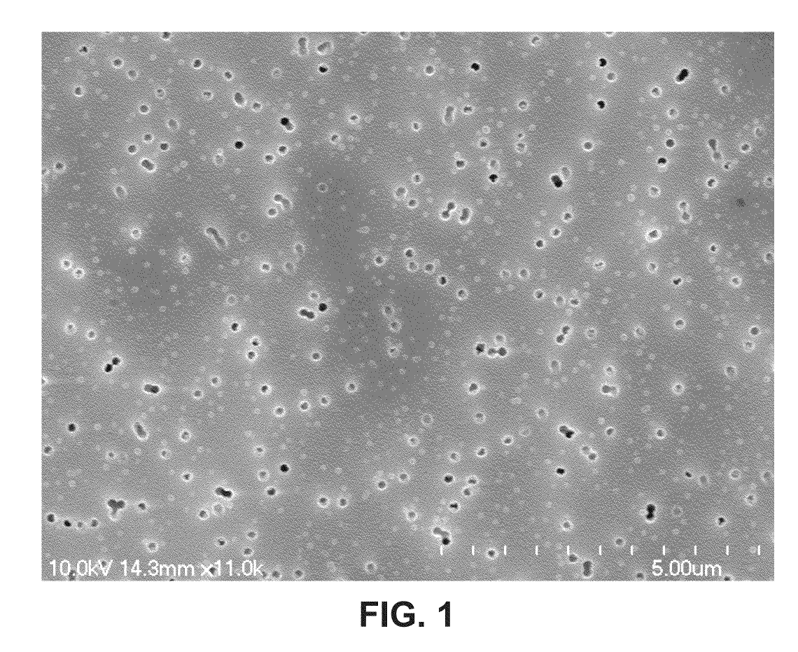

Accordingly, an encapsulated hydrophilic antiretroviral drug including a biodegradable polymeric nanoparticle and a process for fabricating biodegradable polymeric nanoparticles to encapsulate hydrophilic antiretroviral drugs are described. In an implementation, an encapsulated hydrophilic antiretroviral drug including a biodegradable polymeric nanoparticle includes a hydrophilic antiretroviral drug; and a biodegradable polymer polymeric nanoparticle that encapsulates the hydrophilic antiretroviral drug to form a nano-sized encapsulated hydrophilic antiretroviral drug. An example of an encapsulated hydrophilic antiretroviral drug including a biodegradable polymeric nanoparticle is illustrated in FIG. 1.

In an implementation, a process for fabricating biodegradable polymeric nanoparticles to encapsulate hydrophilic antiretroviral drugs includes dissolving at least one of a biodegradable polymer, a poloxamer, or a hydrophilic antiretroviral drug to form a solution; forming a homogenous phase of the solution; emulsifying the solution to form an oil-in-water emulsion; and evaporating the oil-in-water emulsion to form an encapsulated hydrophilic antiretroviral drug including a biodegradable polymeric nanoparticle.

In an implementation, a process for fabricating biodegradable polymeric nanoparticles to encapsulate tenofovir disoproxil fumarate includes dissolving a polylactic-co-glycolic acid (PLGA), a poloxamer, and tenofovir disoproxil fumarate in ethyl acetate and ethanol to form a solution; forming a homogenous phase of the solution by using an orbital shaker at approximately 40° C.; adding dissolved docusate sodium to the solution; emulsifying the solution with water using an ice bath and a sonicator to form an oil-in-water emulsion; and evaporating the ethyl acetate and the ethanol from the oil-in-water emulsion to form a biodegradable polylactic-co-glycolic acid (PLGA) encapsulated tenofovir disoproxil fumarate nanoparticle.

Some hydrophilic drugs (available as salts), such as tenofovir disoproxil fumarate, atazanavir sulfate, and/or abacavir sulfate can form lipophilic complex with anionic ion-pairing agents like sodium lauryl sulfate, sodium deoxycholate, monoammonium glycyrrhizinate, and/or docusate sodium (e.g., dioctyl sodium sulfosuccinate or Aerosol OT) by means of electrostatic interactions. The formation of a lipophilic complex does not involve any chemical modification of the drugs. In the present disclosure, increased encapsulation of the hydrophilic antiretroviral drugs into nanocarriers is disclosed.

### ARV Drugs

In implementations, an encapsulated hydrophilic antiretroviral drug particle can comprise one or more antiretroviral (ARV) drugs. ARV drugs are broadly classified by the phase of the retrovirus life-cycle the drug inhibits. Classes of ARV drugs can include entry inhibitors, CCR5 receptor antagonists, nucleoside reverse transcriptase inhibitors, nucleotide reverse transcriptase inhibitors, non-nucleoside reverse transcriptase inhibitors, protease inhibitors, integrase inhibitors, and maturation inhibitors. Conditions which can be inhibited, prevented or treated with an ARV drug, and thus a composition of the present disclosure, include all conditions associated with HIV, including, but not limited to HIV-1 and HIV-2 infections, and other pathogenic retroviral infections, including AIDS. Management of HIV/AIDS can rely on the use of two or more ARV drugs taken in combination. In some embodiments, ARV drugs can inhibit a retrovirus. In other embodiments, the retrovirus is HIV. In alternative embodiments, ARV drugs can inhibit other viruses. For instance, a 1% tenofivir vaginal gel has been shown to inhibit herpes simplex virus-2 transmission. The technology herein is not limited to inhibiting retroviruses in general, or HIV specifically.

The ARV drugs can be selected from same or different class of ARV drugs. Non-limiting examples of ARV drug classes can include nucleoside reverse transcriptase inhibitors, nucleotide reverse transcriptase inhibitors, non-nucleoside reverse transcriptase inhibitors, protease inhibitors, integrase inhibitors, fusion inhibitors, entry inhibitors (CCR5 co-receptor antagonists) and maturation inhibitors. Non-limiting examples of nucleoside reverse transcriptase inhibitors can include zidovudine, didanosine, stavudine, zalcitabine, abacivir, emtricitabine, and lamivudine. Non-limiting examples of nucleotide reverse transcriptase inhibitors can include tenofovir. Non-limiting examples of non-nucleoside reverse transcriptase inhibitors can include efavirenz (EFV), rilpivirine, etravirine, nevirapine, and delaviradine. Non-limiting examples of protease inhibitors can include HIV protease inhibitors, such as atazanavir, darunavir, indinavir, amprenavir, tipranavir, ritonavir, saquinavir, lopinavir, and nelfinavir. Non-limiting examples of integrase inhibitors can include raltegravir (RAL), elvitegravir dolutegravir. A non-limiting example of a fusion inhibitor can include enfuviritide. A non-limiting example of an entry inhibitor can include mariviroc and cellulose acetate phthalate (CAP). Non-limiting examples of maturation inhibitors can include bevirimat.

In one embodiment, the particle comprises one ARV drug. In another embodiment, the particle comprises two ARV drugs. In yet another embodiment, the particle comprises three ARV drugs. In a different embodiment, the particle comprises four or more ARV drugs. In an exemplary embodiment, the particle comprises two or more ARV drugs selected from the same class. In an alternative embodiment, the particle comprises two or more ARV drugs selected from different classes. In yet another embodiment, the particle comprises two or more ARV drugs, wherein at least ARV drugs are selected from the same class and at least one ARV drug is selected from a different class. In yet another embodiment, the ARV drug is selected from the group consisting of efavirenz, raltegravir, cellulose acetate phthalate, tenofovir, emtricitabine, and combination thereof. Non-limiting examples of ARV drug combinations can include efavirenz plus cellulose acetate phthalate, efavirenz plus raltegravir, and tenofovir plus emtricitabine. In specific implementations, the ARV drug can include at least one of tenofovir disoproxil fumarate, abacavir sulfate, or atazonavir sulfate.

The ARV drug can be associated with the surface of, directly or indirectly conjugated to, encapsulated within, surrounded by, dissolved in, or dispersed throughout a polymeric matrix. The phrase “loaded into”, “loaded onto”, “incorporated into”, or “included in” are used interchangeably to generally describe the association of the ARV drug with the particle without imparting any further meaning as to where or how the ARV drug is associated with the particle. The biochemical properties of the ARV drug can influence the method by which the ARV drug is included in the particle. For instance, a drug's hydrophilicity (as measured by its log P value at ph 7.4) can be used to guide such a decision, as the drug's hydrophilicity can influence the amount of drug that can be encapsulated within a particle. In some embodiments, a hydrophilic drug with a negative log P value at pH 7.4 can be encapsulated within the polymeric mixture, directly or indirectly conjugated to the polymeric mixture, or a combination thereof. In some embodiments, a lipophilic drug with a positive log P value at pH 7.4 can be encapsulated within the polymeric mixture, directly or indirectly conjugated to the polymeric mixture, or a combination thereof. In yet other embodiments, a combination of one or more lipophilic drugs and one or more hydrophilic drugs can be encapsulated within the polymeric mixture, directly or indirectly conjugated to the polymeric mixture, or a combination thereof.

Methods of including the ARV drug in the nanoparticle are described in more detail herein. The amount of each agent present in a particle (entrapment efficiency) can be at least about 10% to as high as about 98% w/w). In some embodiments, the entrapment efficiency can be about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 98% (w/w). Similar to how the biochemical properties of an ARV drug can affect how the ARV drug is loaded into a particle, the biochemical properties can also affect the entrapment efficiency. In some embodiments, wherein the particle comprises two or more ARV drugs, the entrapment efficiency for each ARV drug can be similar. For example, the entrapment efficiency for each of the two or more ARV drugs can be at least about 10% but no greater than about 50% (w/w). In another example, the entrapment efficiency for each of the two or more ARV drugs can be at least about 50% but no greater than about 98% (w/w). In an alternative embodiment, wherein the particle comprises two or more ARV drugs, the entrapment efficiency for each ARV drug can be different. For example, the entrapment efficiency for at least one ARV drug can be at least about 10% but no greater than about 50% (w/w), and the entrapment efficiency for at least one other ARV drug can be at least about 50% but no greater than about 98% (w/w).

### Nanoparticle

The composition (e.g., encapsulated hydrophilic antiretroviral drug) comprises a nanoparticle, the nanoparticle further comprising a polymer and at least one ARV drug. The term “particle,” “nanoparticle,” “biodegradable polymeric nanoparticle,” or the abbreviation “NP” for nanoparticle, as used herein, can refer to particles between 10 and 1000 nanometers (nm) in diameter and are used interchangeably. In these embodiments, the ARV drug can be incorporated into a suitable particle (or nanoparticle) to aid in the delivery of the drug to target cells, to increase the stability of the composition, to minimize potential toxicity of the composition, and/or a combination thereof. A variety of nanoparticles are suitable for delivering an ARV drug.

The size of the particle can influence the ability of the particle to rapidly penetrate through vaginal mucus. For instance, the nanoparticle can have small particle size for successful vaginal delivery. In some embodiments, the diameter of a nanoparticle can be at least 10 nm, at least 20 nm, at least 30 nm, at least 40 nm, at least 50, at least 60 nm, at least 70 nm, at least 80 nm, at least 90 nm, or at least 100 nm. In other embodiments, the particle can be greater than about 100 nm in diameter. For example, the diameter of the nanoparticle can be at least 110 nm, at least 120 nm, at least 130 nm, at least 140 nm, at least 150 nm, at least 160 nm at least 170 nm, at least 180 nm, at least 190 nm, or at least 200 nm. In an exemplary embodiment, the nanoparticle can be less than 220 nm in diameter. In still other embodiments, the particle can be less than about 100 nm in diameter.

In some embodiments, the particle can have a surface charge that is positive or negative. For example, in certain embodiments where a nanoparticle has a negative surface charge, the surface charge can be at least −40 millivolts (mV), at least −35 mV, at least −30 mV, at least −25 mV, at least −20 mV, no greater than −10 mV, no greater than −15 mV, no greater than −20 mV, no greater than −25 mV, or any combination thereof. In an exemplary embodiment, a nanoparticle can have a negative surface charge of at least −30 mV to no greater than −10 mV. In other embodiments wherein a nanoparticle has a positive surface charge, the surface charge can be at least 2 millivolts (mV), at least 15 mV, at least 20 mV, at least 25 mV, or at least 30 mV, no greater than 40 mV, no greater than 35 mV, no greater than 30 mV, no greater than 25 mV, or any combination thereof.

In some embodiments, the particle can have an osmolarity of less than about 1000 mOsm/kg. In other embodiments, the particle can have an osmolarity less than about 500 mOsm/kg. For example, the particle can have an osmolarity of about 50 mOsm/kg, about 100 mOsm/kg, about 150 mOsm/kg, about 200 mOsm/kg, about 250 mOsm/kg, about 300 mOsm/kg, about 350 mOsm/kg, about 400 mOsm/kg, about 410 mOsm/kg, about 420 mOsm/kg, about 430 mOsm/kg, about 440 mOsm/kg, about 450 mOsm/kg, about 460 mOsm/kg, about 470 mOsm/kg, about 480 mOsm/kg, or about 490 mOsm/kg. In another embodiment, the particle can have an osmolarity of at least 500 mOsm/kg to no greater than 1000 mOsm/kg. For example, the particle can have an osmolarity of about 500 mOsm/kg, about 600 mOsm/kg, about 700 mOsm/kg, about 800 mOsm/kg, about 900 mOsm/kg, or about 1000 mOsm/kg.

### Biodegradable Polymer

Each particle can include one or more biodegradable polymers. An example of such a particle comprising a biodegradable polymer and methods of making the particle is disclosed in patent application publication number US 2011/0236437, which is incorporated herein by reference in its entirety. Briefly, a “polymer,” as used herein, is given its ordinary meaning as used in the art, i.e., a molecular structure including one or more repeat units (monomers), connected by covalent bonds. The repeat units can all be identical, or in some cases, there can be more than one type of repeat unit present within the polymer. A polymer can be natural (e.g., biologically derived) or unnatural (e.g., synthetically derived). Polymers can be homopolymers or copolymers including two or more monomers. In teens of sequence, copolymers can be random, block, or can include a combination of random and block sequences. If more than one type of repeat unit is present within the polymer, then the polymer is said to be a “copolymer.” It is to be understood that in any aspect employing a polymer, the polymer can be a copolymer.

A biodegradable polymer is able to degrade, chemically and/or biologically, within a physiological environment, such as within the body. For instance, the polymer can be one that hydrolyzes spontaneously upon exposure to water (e.g., within a subject), or degrades upon exposure to heat (e.g., at temperatures of 42° C.). Degradation of a polymer can occur at varying rates, depending on the polymer or copolymer used. For example, the half-life of the polymer (the time at which 50% of the polymer is degraded into monomers and/or other nonpolymeric moieties) can be on the order of days or weeks, depending on the polymer. The polymers can be biologically degraded, e.g., by enzymatic activity or cellular machinery. In some cases, the polymers can be broken down into monomers and/or other nonpolymeric moieties that cells can either reuse or dispose of without significant toxic effect on the cells (for example, polylactide can be hydrolyzed to form lactic acid, polyglycolide can be hydrolyzed to form glycolic acid, etc.).

In some embodiments, the biodegradable polymer can be a natural polymer. In other embodiments, biodegradable the polymer can be a synthetic polymer. Non-limited examples of natural and synthetic polymers useful in the preparation of biodegradable particles can include carbohydrates such as alginate, cellulose, polyhydroxyalkanoates, polyamides, polyphosphazenes, polypropylfumarates, polyethers, polyacetals, polycyanoacrylates, biodegradable polyurethanes, polycarbonates, polyanhydrides, polyhydroxyacids. poly(ortho esters), and polyesters. Non-limiting examples of polyesters can include polymers including, but not limited to, polycaprolactone, or copolymers including, but not limited to, lactic acid and glycolic acid units, such as poly(lactic acid-coglycolic acid) and poly(lactide-co-glycolide), collectively referred to herein as “PLGA”; and homopolymers including glycolic acid units, and lactic acid units, such as poly-L-lactic acid, poly-D-lactic acid, poly-D,L-lactic acid, poly-L-lactide, poly-D-lactide, and poly-D,L-lactide. In some embodiments, the polymer can be PLGA. In another embodiment, the polymer can be polycaprolactone. In yet another embodiment, the polymer can be cellulose acetate phthalate. In some embodiments, the polymeric nanoparticles can include tenofovir disoproxil fumarate (with high encapsulation efficiency) and can be freeze-dried to convert into a solid powder with the help of suitable cryoprotectants.

### Thermosensitive Gel

In implementations, the encapsulated hydrophilic antiretroviral drugs can be incorporated into a thermosensitive gel base, which may gel upon heating. As used herein, the term “gels” can refer to any process by which a composition changes from a solution into a gel (i.e. undergoes a sol-gel transition), and the term “thermosensitive gel” can refer to a polymeric system that undergoes a sol-gel transition due to temperature. Generally speaking, the composition is a thermosensitive gel that is a solution at room temperature but forms a gel once delivered inside the body. For example, the gel can be a citric acid based aqueous substance with a pH of about 4.5 to be compatible with a female reproductive track pH. One skilled in the art will appreciate that “room temperature” will vary depending on the local climate. In an exemplary embodiment, the thermosensitive gel remains a liquid in subtropical and tropical countries or in the zone IV as classified by the guidelines from the International Conference on Harmonisation of Technical Requirements for Registration of Pharmaceuticals for Human Use. For example, the thermogelation temperature can be between 30-35° C. In some embodiments, the thermogelation temperature can be about 30.0° C., 30.1° C., 30.2° C., 30.3° C., 30.4° C., 30.5° C., 30.6° C., 30.7° C., 30.8° C., 30.9° C., 31° C., 31.1° C., 31.2° C., 31.3° C., 31.4° C., 31.5° C., 31.6° C., 31.7° C., 31.8° C., 31.9° C., 32° C., 32.1° C., 32.2° C., 32.3° C., 32.4° C., 32.5° C., 32.6° C., 32.7° C., 32.8° C., 32.9° C., 33° C., 33.1° C., 33.2° C., 33.3° C., 33.4° C., 33.5° C., 33.6° C., 33.7° C., 33.8° C., 33.9° C., 34° C., 34.1° C., 34.2° C., 34.3° C., 34.4° C., 34.5° C., 34.6° C., 34.7° C., 34.8° C., 34.9° C., or 35° C. In other embodiments, the thermogelation temperature can be about 30 to about 33° C.

A variety of polymers can undergo sol-gel transitions due to temperature and may be implemented. In some embodiments, the thermosensitive polymer is synthetic or naturally derived. In other embodiments, the thermosensitive polymer can also have mucoadhesive or bioadhesive properties. In still other embodiments, the thermosensitive polymer does not have mucoadhesive or bioadhesive properties. In yet other embodiments, thermosensitive polymers are combined with mucoadhesive polymers. In implementations, the thermosensitive polymer and its gel can be biocompatible when used for pharmaceutical applications. As used herein, “biocompatible” means the polymer does not typically induce significant inflammation and/or acute rejection of the polymer by the immune system when introduced into a living subject. Non-limiting examples of suitable thermosensitive polymers can include chitosan-based copolymers, N-isopropylacrylamide copolymers, poly(ethylene oxide)/poly(propylene oxide) block copolymers, poly(ethylene glycol)/poly(D,L-lactide-co-glycolide) block copolymers, and poloxamers. Poloxamers are nonionic triblock copolymers composed of a central hydrophobic chain of polyoxypropylene (poly(propylene oxide)) flanked by two hydrophilic chains of polyoxyethylene (poly(ethylene oxide)). Poloxamers are also known by the trade name Pluronics and Kolliphor. In some embodiments, the polymeric system can be comprised of a Pluronic or a mix of Pluronics. In other embodiments, the polymeric system can include Pluronic F127 and/or Pluronic F68. The ratio of Pluronic F127 to Pluronic F68 can vary. In some embodiments, the ratio of Pluronic F127 to Pluronic F68 (% weight/volume) is between about 20%:1% to about 18%:1%. In other embodiments the ratio of Pluronic F127 to Pluronic F68 (% weight/volume) is between about 20%:2% to about 18%:2%. In still other embodiments, the ratio of Pluronic F127 to Pluronic F68 (% weight/volume) is 20%:1%.

Methods of developing a thermosensitive gel can include mixing known quantities of one or more thermosensitive polymers in solution, and determining the thermogelation point of the gel and dynamic viscosity. Pharmaceutical formulations and other compositions can be incorporated by solution mixing. Methods of developing a thermosensitive gel are described in more detail herein.

### Additional Components

The particles can also optionally comprise polypeptides, small organic molecules, polysaccharides, polynucleotides, natural products, synthetic compounds, chemical compounds, or a combination thereof. In one embodiment, a particle can optionally comprise a substance that improves the mucous-penetrating ability of the particle. In another embodiment, a particle can optionally comprise a targeting molecule. A targeting molecule is able to bind a biological entity, such as a membrane or cell surface receptor. Suitable targeting molecules are known in the art. In an exemplary embodiment, a particle can optionally comprise a stabilizer. A non-limiting example of a stabilizer can include Pluronic F127.

### TDF Nanoparticle Characterization

In one study, nanoparticle characterization after tenofovir disoproxil fumarate (TDF) incorporation into nanoparticles (NPs) demonstrated mean (+SD) particle size of TDF-NPs was 68.2±2.9 nm and surface charge was −4.5±0.9 mV. The polydispersity of the NP size for the TDF NPs averaged <10% from the mean particle size. The entrapment efficiency (EE) of TDF in TDF-NPs (in absence of ion-pairing agents) was 16.1%. However, in the presence of ion-pairing agent, mean EE of TDF in TDF-NPs increased to 95.8%. The fabrication process of TDF NPs was optimized to obtain two different concentrations of TDF (0.1% and 0.5% w/v) for use in the prevention of HIV-1 using the thermosensitive (TMS) gel as a vaginal delivery system in the humanized BLT mouse model. FIG. 1 shows an electron microscope image of the nanoparticles.

### TDF Nanoparticles Are Not Cytotoxic In Vitro

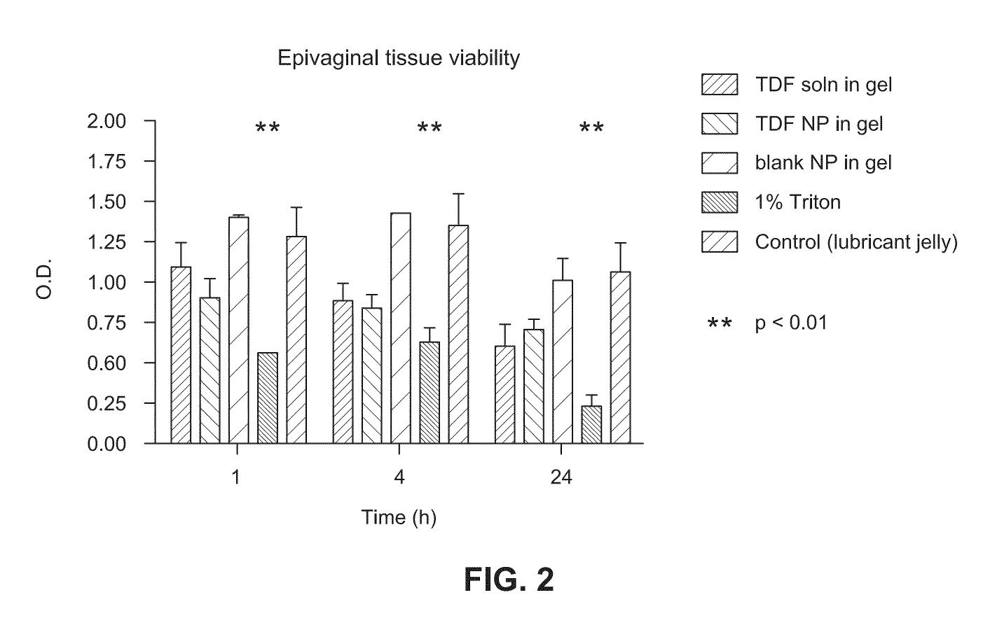

In multiple cell-lines and primary cells, TDF NPs were not more cytotoxic compared to TDF in solution in vitro. In Epivaginal™ tissue cultures, 1% Triton (positive control) produced the most cytotoxicity (p<0.01; ANOVA; See FIG. 2). TDF NPs in TMS gel and TDF solution in TMS gel were not cytotoxic compared to the other variables and control vaginal lubricant jelly.

### Hu-BLT PrEP Experiments

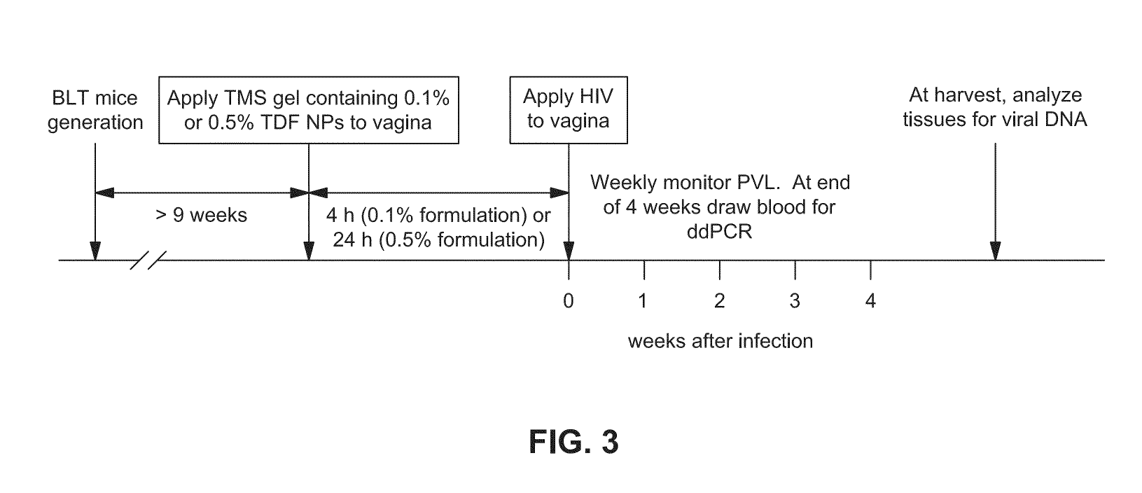

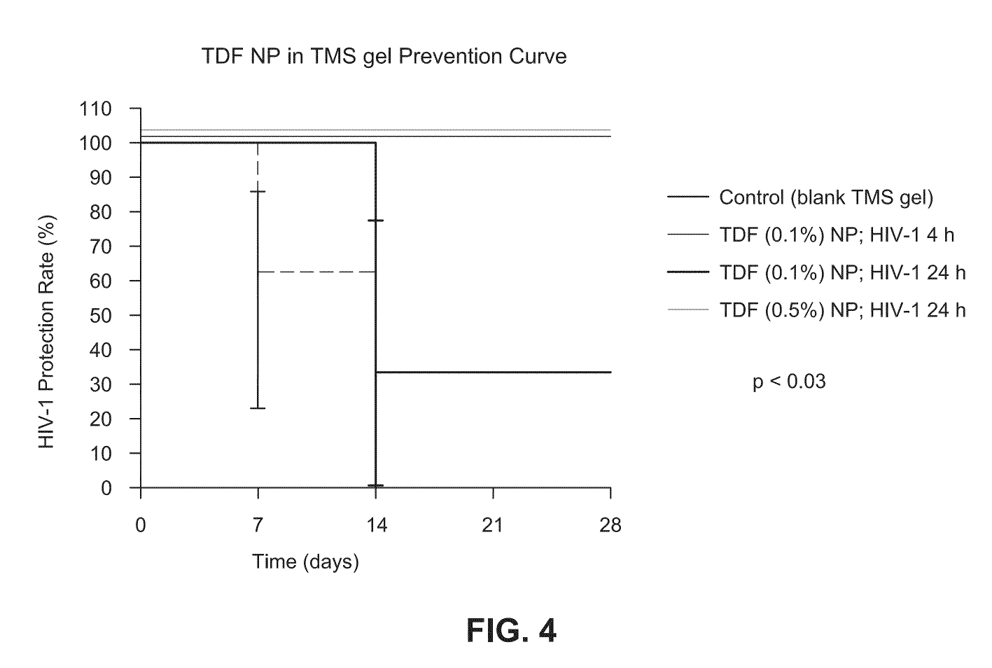

One prevention experimental study design using hu-BLT mice is illustrated in FIG. 3. Control hu-BLT mice (n=8) that received blank TMS gel intravaginally and were challenged with the same dose and strains of HIV-1 4 h later all contracted HIV-1. Treated hu-BLT mice that received 0.1% TDF NP (n=4) and were challenged with HIV-1 4 h later, were all protected from HIV-1 infection. 33% of the Hu-BLT mice (n=3) that received 0.1% TDF NP and were challenged 24 h later, mice were protected from HIV-1 infection. 100% of the Hu-BLT mice (n=4) that received 0.5% TDF NP and challenged with HIV-1 24 h later, were protected from HIV-1 infection (FIG. 4). Taking these results in total, TDF NPs in TMS gel given at 4 h (0.1%) and 24 h (0.5%) demonstrated significant (p<0.03; Mantel-Cox test) protection from HIV-1 vaginal transmission when delivered as a thermosensitive vaginal gel. HIV-1 vRNA examined using ISH were negative in both vaginal and spleen tissues of the hu-BLT mice that had undetectable PVL. These results prove that compared to control mice, 0.5% TDF NPs were able to protect female Hu-BLT mice from HIV-1 challenge over a prolonged time frame using an intravaginal route of transmission.

### Methods of Use

The composition can be administered prophylactically. Prophylactic use prior to exposure (pre-exposure prophylaxis) is a prevention method in which people who are not virally infected take medication to reduce their risk of infection in the event of exposure to the virus following sexual intercourse. However, a prophylaxis can also be effective shortly before or shortly after exposure to the virus following sexual intercourse. In some embodiments, the composition can be administered peri-coital. In other embodiments, the composition can be administered post-coital. In still other embodiments, the composition can be administered pre-coital.

The amount of time that can elapse between administration of the composition and viral exposure can vary depending on variables such as the ARV drug or combination of ARV drugs in the composition as well as the amount (or dose) of the ARV drug or combination of ARV drugs. Coitally-dependent gels require insertion within a brief window of time before sexual intercourse. In some implementations, the composition can be developed as a coitally-independent gel. In some embodiments, the composition can be administered about 12 hours prior to exposure. In other embodiments, the composition can be administered about 1 day, about 2 days, about 3 days, about 4 days, about 5 days, about 6 days, about 7 days, about 8 days, about 9 days, about 10 days, about 11 days, about 12 days, about 13 days, or about 14 days prior to exposure. In still other embodiments, the composition can be administered up to and including about 1 week prior to exposure.

The composition can be administered vaginally or rectally. The composition can be provided as dry powder (e.g., freeze-dried), potentially to be reconstituted, a liquid and/or as a gel. In one method of administration, the composition can be maintained at a temperature of less than the thermogelation temperature to keep it in a liquid state and administered into the vagina or the anus, where it forms a gel when at the temperature of the human body. In another method of administration, the composition can be brought to a temperature on or about its thermogelation temperature to form a gel and then administered as a gel into the vagina or the anus. In implementations, a syringe, or any other device known in the art, can be used for injection. If it is to be injected as a gel, the composition may be warmed in the syringe to form a gel and injected from the syringe as a gel to the affected site. Alternatively, the composition may be in the gel state when it is loaded into the syringe. In still another method of administration, the composition may be brought to a temperature on or about its thermogelation temperature to form a gel in the presence of a device that can be inserted into the vagina (e.g. intravaginal rings or diaphragms), such that the gel forms on the device. Alternatively, the composition can also be formulated in rectal compositions such as suppositories or retention enema, using, e.g., suppository bases such as cocoa butter or other glycerides.

### Short-Term In-Vitro Prophylaxis

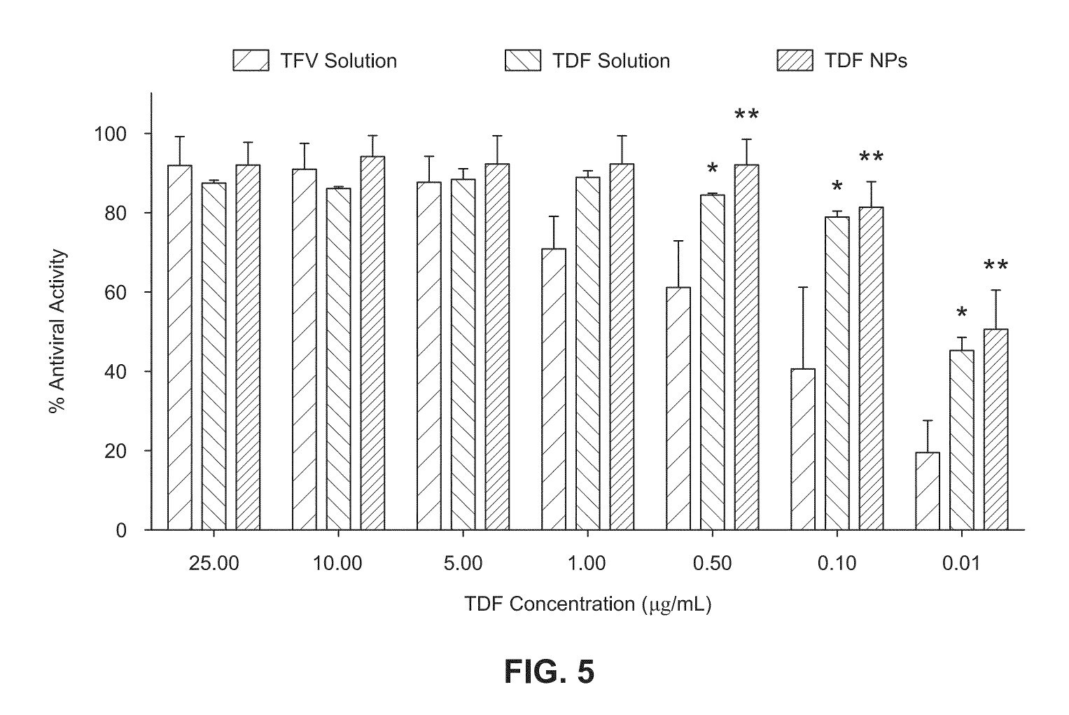

In one study, short-term (1 day pre-treatment) in vitro prophylaxis of tenofovir (TFV) solution, TDF solution and TDF-NPs against HIV-1_{NL4-3} was determined using TZM-bl HIV indicator cells. Briefly, TZM-bl cells were seeded in 24-well plates at a density of 1×10⁴ cells per well. After 24 h, incubation, the cells were treated with different concentrations of TFV solution, TDF solution and TDF-NPs (concentration range: 25 μg/ml to 10 ng/mL). After 24 h, media was removed from all the wells and replaced with fresh media. On the following day, the cells were inoculated with HIV-1_{NL4-3} virus (25 μl) for 4 h. The cells were washed after the 4 h and incubated for 48 h. Cells were lysed and BrightGLO™ (Promega, Madison, Wis.) assay was used to measure the luminescence obtained with different treatments. TZM-bl cells without HIV infection and HIV-infected cells with no treatment served as controls. Luminescence was determined based on relative luminescence units (RLU). The % protection from HIV-1 infection was calculated using following formula: % protection from HIV-1 infection=[(L_{untreated}−L_{treated})/L_{untreated}]×100, where L_{untreated} is the luminescence of HIV-infected TZM-bl cells without treatment and L_{treated} is the luminescence of HIV-infected TZM-bl cells treated with TFV solution, TDF solution or TDF-NPs. Results of these experiments are shown in FIG. 5 and show that a greater TDF concentration results in greater antiviral activity.

### Fabrication of Tenofovir Disoproxil Fumarate Loaded PLGA Nanoparticles

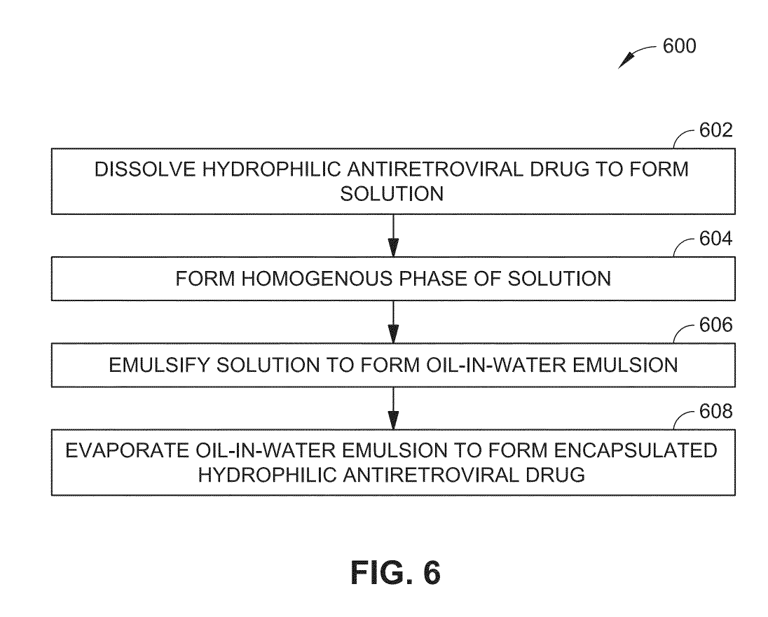

The following discussion describes example techniques for fabricating a hydrophilic antiretroviral drug using a nanocarrier. FIG. 6 depicts an example process 600 for fabricating a hydrophilic antiretroviral drug using a nanocarrier.

As shown in FIG. 6, at least one of a biodegradable polymer, a poloxamer, or a hydrophilic antiretroviral drug is dissolved to form a solution (Block 602). In one example, Resomer 752H (acid terminated PLGA with lactic to glycolic acid ratio of 75:25) can be used. Resomer 752 H (500 mg), Pluronic F127 (Poloxamer 407; 250 mg) and tenofovir disoproxil fumarate (50 mg) can be dissolved in 2.5 mL of ethyl acetate and 0.5 mL of ethanol (organic phase). Next, a homogenous phase of the solution is formed (Block 604). The organic phase (in a scintillation vial) can be shaken on an orbital shaker maintained at 40° C. to form the homogenous phase. In some embodiments, docusate sodium (75 mg) may be dissolved in 0.5 mL of ethyl acetate, and the solution can be transferred to the organic phase. Then, the solution is emulsified to form an oil-in-water solution (Block 606). The organic phase can be emulsified in 10 mL of ultrapure water using a probe sonicator (UP100H; Hielscher USA, Inc., NJ, USA; amplitude: 80% and intensity: 0.8) for 15 min in an ice bath. Next, the oil-in-water emulsion is evaporated to form an encapsulated hydrophilic antiretroviral drug (Block 608). The resultant oil-in-water emulsion can be transferred to a 50 mL beaker and stirred at 700 rpm for 3 h using a magnetic stirrer to evaporate the ethyl acetate and ethanol. Particle size, polydispersity index, and surface charge of resulting tenofovir disoproxil fumarate nanoparticles were measured using dynamic light scattering (ZetaPlus instrument, Brookhaven Instruments Corp, NY, USA). This example is non-limiting, and it is contemplated that similar and/or other equipment and/or methods may be utilized.

In some specific embodiments, tenofovir disoproxil fumarate loaded nanoparticles may also be fabricated in the absence of anionic ion-pairing agent (docusate sodium).

In a specific embodiment, the encapsulation of tenofovir disoproxil fumarate in nanoparticles were prepared with and without anionic ion-pairing agents, and the encapsulation efficiency of tenofovir disoproxil fumarate in nanoparticles was determined by a reverse-phase HPLC method. In this specific embodiment, the tenofovir disoproxil fumarate nanoparticles had size of 68.2±2.9 nm, polydispersity index of 0.17±0.05 and surface charge −4.5±0.9 mV. The encapsulation efficiency of tenofovir disoproxil fumarate in the nanoparticles prepared without using anionic ion-pairing agent was 16.1±2.3%. However, in the presence of anionic ion-pairing agent docusate sodium, the encapsulation efficiency of tenofovir disoproxil fumarate in the nanoparticles increased to 95.8±0.7%. Similar results were observed with another anionic ion-pairing agent, sodium lauryl sulfate.

In some embodiments, the polymeric nanoparticles containing tenofovir disoproxil fumarate (with high encapsulation efficiency) can be freeze-dried to convert into a solid powder with the help of suitable cryoprotectants. These nanoparticles can be used for long-term prevention and treatment of HIV-1 infections on oral or subcutaneous administration.

### CONCLUSION

Although the subject matter has been described in language specific to structural features and/or process operations, it is to be understood that the subject matter defined in the appended claims is not necessarily limited to the specific features or acts described above. Rather, the specific features and acts described above are disclosed as example forms of implementing the claims.

What is claimed is:

1. An encapsulated hydrophilic antiretroviral drug including a biodegradable polymeric nanoparticle, comprising:
a hydrophilic antiretroviral drug; and
a biodegradable polymer polymeric nanoparticle that encapsulates the hydrophilic antiretroviral drug to form a nano-sized encapsulated hydrophilic antiretroviral drug.

2. The encapsulated hydrophilic antiretroviral drug of claim 1, where the hydrophilic antiretroviral drug includes at least one of tenofovir disoproxil fumarate, abacavir sulfate, or atazanavir sulfate.

3. The encapsulated hydrophilic antiretroviral drug of claim 1, where the biodegradable polymer polymeric nanoparticle includes polylactic-co-glycolic acid (PLGA).

4. The encapsulated hydrophilic antiretroviral drug of claim 1, where the biodegradable polymer polymeric nanoparticle includes a US Food and Drug Administration (US FDA) approved biodegradable polymer.

5. The encapsulated hydrophilic antiretroviral drug of claim 1, where the nano-sized encapsulated hydrophilic antiretroviral drug is a freeze-dried solid powder.

6. The encapsulated hydrophilic antiretroviral drug of claim 1, further comprising a thermosensitive gel base that forms a tenofovir disoproxil fumarate gel.

7. A process for fabricating biodegradable polymeric nanoparticles to encapsulate hydrophilic antiretroviral drugs, comprising:
dissolving at least one of a biodegradable polymer, a poloxamer, or a hydrophilic antiretroviral drug to form a solution;
forming a homogenous phase of the solution;
emulsifying the solution to form an oil-in-water emulsion; and
evaporating the oil-in-water emulsion to form an encapsulated hydrophilic antiretroviral drug including a biodegradable polymeric nanoparticle.

8. The process for fabricating biodegradable polymeric nanoparticles of claim 7, wherein the biodegradable polymer includes polylactic-co-glycolic acid (PLGA).

9. The process for fabricating biodegradable polymeric nanoparticles of claim 7, wherein the hydrophilic antiretroviral drug includes tenofovir disoproxil fumarate.

10. The process for fabricating biodegradable polymeric nanoparticles of claim 7, wherein the hydrophilic antiretroviral drug includes at least one of abacavir sulfate or atazanavir sulfate.

11. The process for fabricating biodegradable polymeric nanoparticles of claim 7, wherein dissolving at least one of the biodegradable polymer, the poloxamer, or the hydrophilic antiretroviral drug includes using at least one of ethyl acetate or ethanol.

12. The process for fabricating biodegradable polymeric nanoparticles of claim 7, wherein forming the homogenous phase of the solution includes shaking the solution.

13. The process for fabricating biodegradable polymeric nanoparticles of claim 7, wherein emulsifying the solution includes sonicating the solution with water in an ice bath.

14. The process for fabricating biodegradable polymeric nanoparticles of claim 7, wherein evaporating the oil-in-water emulsion includes evaporating at least one of ethyl acetate or ethanol.

15. The process for fabricating biodegradable polymeric nanoparticles of claim 7, wherein the encapsulated hydrophilic antiretroviral drug including the biodegradable polymeric nanoparticle includes tenofovir disoproxil fumarate nanoparticles.

16. The process for fabricating biodegradable polymeric nanoparticles of claim 7, further comprising:
combining an anionic ion-pairing agent with the solution to form a lipophilic complex in-situ that is encapsulated into the biodegradable polymeric nanoparticles.

17. The process for fabricating biodegradable polymeric nanoparticles of claim 16, wherein the anionic ion-pairing agent includes at least one of sodium lauryl sulfate, sodium deoxycholate, monoammonium glycyrrhizinate, or docusate sodium.

18. The process for fabricating biodegradable polymeric nanoparticles of claim 7, further comprising:
freeze-drying the encapsulated hydrophilic antiretroviral drug including the biodegradable polymeric nanoparticle with a cryoprotectant to form a solid powder.

19. The process for fabricating biodegradable polymeric nanoparticles of claim 7, further comprising:
incorporating the encapsulated hydrophilic antiretroviral drug including the biodegradable polymeric nanoparticle into a thermosensitive gel base.

20. A process for fabricating biodegradable polymeric nanoparticles to encapsulate tenofovir disoproxil fumarate, comprising:
dissolving a polylactic-co-glycolic acid (PLGA), a poloxamer, and tenofovir disoproxil fumarate in ethyl acetate and ethanol to form a solution;
forming a homogenous phase of the solution by using an orbital shaker at approximately 40° C.;
adding dissolved docusate sodium to the solution;
emulsifying the solution with water using an ice bath and a sonicator to form an oil-in-water emulsion; and
evaporating the ethyl acetate and the ethanol from the oil-in-water emulsion to form a biodegradable polylactic-co-glycolic acid (PLGA) encapsulated tenofovir disoproxil fumarate nanoparticle.
